(19) United States
(12) Patent Application Publication
(10) Pub. No.: US 2014/0081162 A1
(43) Pub. Date: Mar. 20, 2014

(54) METHOD AND SYSTEM FOR ST MORPHOLOGY DISCRIMINATION UTILIZING REFERENCE MORPHOLOGY TEMPLATES

(71) Applicant: PACESETTER, INC., Sylmar, CA (US)

(72) Inventors: Jay Snell, Studio City, CA (US); Carol Hudgins, Crestwood, KY (US); Kathleen Kresge, Minneapolis, MN (US)

(73) Assignee: PACESETTER, INC., Sylmar, CA (US)

(21) Appl. No.: 13/622,919

(22) Filed: Sep. 19, 2012

(51) Int. Cl. A61B 5/0468; A61B 5/0456

(52) U.S. Cl. 600/516

(57) ABSTRACT

Methods and systems are provided that utilize reference morphology templates as morphology based filters to reduce false or inappropriate ST episode detections when an ST shift episode is otherwise diagnosed. The methods and systems provide ST morphology discrimination. The methods and systems sense cardiac signals of a heart, obtain a reference morphology template based on at least one baseline cardiac signal associated with a normal physiology waveform, and identify a potential ST segment shift from the cardiac signals. The methods and systems compare the cardiac signals to the reference morphology template to derive a morphology indicator representing a degree to which the cardiac signals match the reference morphology template; and declare the potential ST segment shift to be an actual ST segment shift based on the morphology indicator.

### BACKGROUND OF THE INVENTION

Embodiments of the present invention generally relate to morphology discrimination, and more particularly to methods and systems that utilize reference morphology templates to validate ST morphology discrimination.

An implantable medical device is implanted in a patient to monitor, among other things, electrical activity of a heart and to deliver appropriate electrical and/or drug therapy, as required. Implantable medical devices (“IMDs”) include for example, pacemakers, cardioverters, defibrillators, implantable cardioverter defibrillators (“ICD”), and the like. The electrical therapy produced by an IMD may include, for example, pacing pulses, cardioverting pulses, and/or defibrillator pulses to reverse arrhythmias (e.g., tachycardias and bradycardias) or to stimulate the contraction of cardiac tissue (e.g., cardiac pacing) to return the heart to its normal sinus rhythm.

Cardiac ischemia is a condition whereby the heart tissue does not receive adequate amounts of oxygen that is usually caused by a blockage of an artery leading to the heart tissue. Ischemia arises during angina, coronary angioplasty, and any other condition that compromises blood flow to a region of myocardial. When blockage of an artery is sufficiently severe, the cardiac ischemia becomes an acute myocardial infarction (AMI), which is also referred to as a myocardial infarction (MI) or a heart attack.

Many patients at risk of various heart conditions, such as cardiac ischemia, have pacemakers, ICDs, ISCDs, or other medical devices implanted therein. Electrocardiograms (ECG) are useful for diagnosing certain heart conditions, such as ischemia and locating damaged areas within the heart. ECGs are composed of various waves and segments that represent the heart depolarizing and repolarizing. The ST segment represents the portion of the cardiac signal between ventricular depolarization and ventricular repolarization. While P-waves, R-waves, and T-waves may be generally considered features of a surface electrocardiogram (ECG), for convenience and generality, herein the terms R-wave, T-wave, and P-wave are also used to refer to the corresponding internal cardiac signal, such as an intra-cardiac electrogram (IEGM) signal. Techniques have been developed for detecting heart conditions using implanted medical devices by identifying variations in the ST segment from the baseline cardiac signal that occur during ST episodes (e.g. cardiac ischemia). Deviation of the ST segment during an ST episode from a baseline is a result of injury to cardiac muscle, variations in the synchronization of ventricular muscle depolarization, drug or electrolyte influences, or the like. Various morphology discrimination techniques have been proposed that utilize ST segment shifts to identify ST episodes.

However, conventional morphology discrimination techniques declare an unduly false positive ST episode. False positive ST episode declaration may be caused by rate dependent bundle branch blocks, posture-related axis changes of the EGM signal and other non-ST segment related physiologic behavior. Approximately 30% of all false positive detections (FPD) analyzed in the OUS Registry may be a result of ST segment changes that are classified as ST episodes, but in reality are not ST episodes, instead arising from intermittent or rate dependent fascicular block or Bundle Branch Block (BBB). One approach to removing FPDs is to entirely disable the ST monitoring feature for IMDs in patients who manifest conduction abnormalities that cause EGM perturbations that appear as ST episodes, but are not ST episodes. However, it would be preferred to continue use of the ST monitoring feature in the IMD even in such patients.

A need remains for an ST monitoring method and system able to reduce or prevent false positive ST episode detections caused by rate dependent bundle branch blocks, posture-related axis changes of the EGM signal and other non-ST segment related physiologic behavior.

### SUMMARY

In accordance with one embodiment, methods and systems are provided utilizing reference morphology templates as morphology-based filters to reduce false or inappropriate ST episode detections whenever an ST shift episode is otherwise diagnosed.

In accordance with an embodiment, a method is provided for ST morphology discrimination. The method comprises sensing cardiac signals of a heart, obtaining a reference morphology template based on at least one baseline cardiac signal associated with a normal physiology waveform, and identifying a potential ST segment shift from the cardiac signals. The method further comprises comparing the cardiac signals to the reference morphology template to derive a morphology indicator representing a degree to which the cardiac signals match the reference morphology template; and declaring the potential ST segment shift to be an actual ST segment shift based on the morphology indicator.

Optionally, the obtaining operation includes obtaining first and second reference morphology templates, the first reference morphology template based on at least one baseline cardiac signal associated with a normal physiology waveform, the second reference morphology template based on at least one baseline cardiac signal associated with an abnormal physiology waveform. Optionally, the comparing operation compares the cardiac signals to the first and second reference morphology templates to derive first and second morphology indicators, respectively. Optionally, the declaring operation declares the potential ST segment shift to be an actual ST segment shift based on the first and second morphology indicators.

Optionally, the method further comprises collecting a reference morphology template based on sampling of multiple baseline cardiac signals associated with normal physiology waveforms. The sensing, identifying, comparing and declaring operations are repeated for sets of cardiac events, each cardiac event associated with a heart beat. The method further comprises declaring a set of the cardiac events to exhibit ST segment shift when a predetermined number of cardiac events in the corresponding set have morphology indicators indicating a predetermined degree of match with the reference morphology template.

Optionally, the method further comprises collecting multiple reference morphology templates, each of which is associated with a unique corresponding heart rate zone, the identifying, comparing and declaring operations utilizing one of the multiple reference templates corresponding to a present heart rate associated with the cardiac signals sensed. Optionally, the method further comprises identifying a polarity of a dominant R-peak in the cardiac signal, comparing the polarity of the dominant R-peak in the cardiac signal with a polarity of a dominant R-peak in the reference morphology template, the declaring operation based in part on the polarity comparison. Optionally, the comparing operation includes comparing one or more of the following: wave frequency, an area of each QRS segment, a sequence of R-peaks, a number of R-peaks, an amplitude of R-peaks, and a polarity of the R-peaks.

In accordance with an embodiment, a system is provided that comprises an input configured to receive cardiac signals sensed from a heart, an ST episode detection unit configured to monitor the cardiac signals and identify a potential ST segment shift based thereon, and a template acquisition unit configured to obtain a reference morphology template based on at least one baseline cardiac signal associated with a normal physiology waveform. The system further comprises a comparison unit configured to compare the cardiac signals to the reference morphology template to derive a morphology indicator representing a degree to which the cardiac signals match the reference morphology template; and a validation unit configured to declare the potential ST segment shift to be an actual ST segment shift based on the morphology indicator, the ST episode detection unit configured to declare an ST episode when a predetermined number of actual ST segment shifts are validated.

Optionally, the template acquisition unit is configured to obtain first and second reference morphology templates, the first reference morphology template based on at least one baseline cardiac signal associated with a normal physiology waveform, the second reference morphology template based on at least one baseline cardiac signal associated with an abnormal physiology waveform. Optionally, the comparison unit is configured to compare the cardiac signals to the first and second reference morphology templates to derive first and second morphology indicators, respectively. Optionally, the validation unit is configured to declare the potential ST segment shift to be an actual ST segment shift based on the first and second morphology indicators. Optionally, the template acquisition unit is configured to collect a reference morphology template based on sampling of multiple baseline cardiac signals associated with normal physiology waveforms.

Optionally, the sensing, identifying, comparing and declaring functions are repeated for sets of cardiac events, each cardiac event associated with a heart beat. The ST episode detection unit is configured to declare a set of the cardiac events to exhibit ST segment shift when a predetermined number of cardiac events in the corresponding set have morphology indicators indicating a predetermined degree of match with the reference morphology template. Optionally, the template acquisition unit is configured to collect multiple reference morphology templates, each of which is associated with a unique corresponding heart rate zone, the identifying, comparing and declaring operations utilizing one of the multiple reference templates corresponding to a present heart rate associated with the cardiac signals sensed. Optionally, the comparison unit is configured to identify a polarity of a dominant R-peak in the cardiac signal, and compare the polarity of the dominant R-peak in the cardiac signal with a polarity of a dominant R-peak in the reference morphology template, the validation unit configured to validate the actual ST segment shift based in part on the polarity comparison.

Optionally, the comparison unit is configured to compare one or more of the following: wave frequency, an area of each QRS segment, a sequence of R-peaks, a number of R-peaks, an amplitude of R-peaks, and a polarity of the R-peaks.

### BRIEF DESCRIPTION OF THE DRAWINGS

FIG. 1 illustrates an implantable medical device that is coupled to a heart that is utilized in accordance with an embodiment.

FIG. 2 illustrates a block diagram of exemplary internal components of an IMD implemented in accordance with an embodiment.

FIG. 3 illustrates a functional block diagram of an external device implemented in accordance with an embodiment.

FIG. 4 illustrates an example of an ST-MD process that includes validation based on RMTs in accordance with an embodiment.

FIG. 5 illustrates a single cardiac cycle composed of a P-wave, a Q-wave, an R-wave, an S-wave, and a T-wave.

FIG. 6 illustrates a process for acquiring active “Normal” (baseline) and “Abnormal” (reverse) reference morphology templates (RMTs) in accordance with an embodiment.

FIG. 7 illustrates a processing sequence carried out to perform a cross-check validation for potential ST segment shifts in accordance with an embodiment.

FIG. 8 illustrates a processing sequence carried out to perform a cross-check validation for potential ST segment shifts in accordance with an embodiment.

FIG. 9 provides a sectional view of a patient's heart and shows a leadless implantable medical device (LIMD) that may implement the methods described herein.

### DETAILED DESCRIPTION

In the following detailed description, reference is made to the accompanying drawings which form a part hereof, and in which are shown by way of illustration specific embodiments in which the present invention may be practiced. These embodiments, which are also referred to herein as “examples,” are described in sufficient detail to enable those skilled in the art to practice the invention. It is to be understood that the embodiments may be combined or that other embodiments may be utilized, and that structural, logical, and electrical variations may be made without departing from the scope of the present invention. For example, embodiments may be used with a pacemaker, a cardioverter, a defibrillator, leadless implantable medical devices and the like. The following detailed description is, therefore, not to be taken in a limiting sense, and the scope of the present invention is defined by the appended claims and their equivalents. In this document, the terms “a” or “an” are used, as is common in patent documents, to include one or more than one. In this document, the term “or” is used to refer to a nonexclusive or, unless otherwise indicated.

Embodiments are described herein for an ST morphology discrimination (MD) system and method that are utilized to help distinguish a normally conducted intrinsic ventricular beat from one with delayed conduction. For example, delayed conduction may result from right or left bundle branch block (BBB), intra-ventricular conduction delay (IVCD), hemi-block that alters the IEGM morphology and other abnormal physiologic behaviors that cause existing ST segment analysis to be unreliable. In one embodiment, a method and system are provided, having a baseline extraction phase, during which one or more reference morphology templates are collected based on a sample of cardiac signals from multiple heart beats. The templates may include one or more corresponding to normal baseline morphology and one or more corresponding to abnormal or “reverse” morphology.

In at least one embodiment, the ST morphology discrimination method and system store one or more templates at periodic intervals, such as once per week. Separate templates are stored for each of multiple elevated baseline heart rate (HR) zones, in addition to a baseline resting template which is collected or extracted when the patient is in a resting HR zone. The ST-MD method and system then, during an on-going monitor phase, collects new heart beats and compares the QRS morphology of the new or present heart beat to one or more stored morphology templates. The QRS morphologies for new heart beats are compared at regular intervals, such as every 30 seconds, to the templates while the device continues to monitor for ST shifts. Normal heart beats, when compared to the morphology templates, generates a morphology indicator representing a high score of percent match (typically >90%). A high percentage match score indicates a similarity to the “normal” baseline rhythm. A heart beat with delayed conduction (e.g., intermittent BBB), when compared to the morphology templates, generates a morphology indicator having a lower score of percent match. A low percentage match score indicates a dis-similarity to the “normal” baseline rhythm, or a similarity to an “abnormal” rhythm.

The ST morphology method and system can be used as a cross-check once the ST episode monitoring algorithm has detected a ST shift. If a morphology template is not a sufficient match to a normal template, the device would suspend ST monitoring and, thus detection of the ST episode, and return to periodically monitoring of the ST segments. In addition to a morphology template match score, the algorithm may also track the polarity of the dominant R peak as a secondary analysis for scoring.

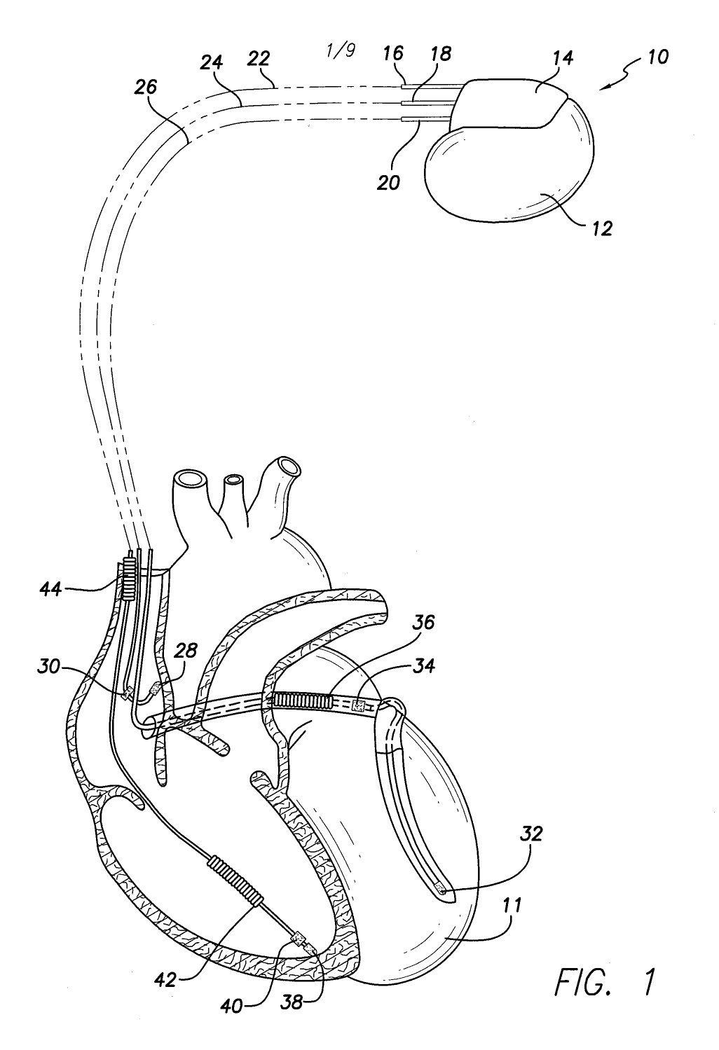

FIG. 1 illustrates an implantable medical device 10 (IMD) that is coupled to a heart 11. The implantable medical device 10 may be a cardiac pacemaker, an implantable cardioverter defibrillator (“ICD”), a defibrillator, or an ICD coupled with a pacemaker implemented in accordance with an embodiment of the present invention. The IMD 10 may be a dual-chamber stimulation device capable of treating both fast and slow arrhythmias with stimulation therapy, including cardioversion, defibrillation, and pacing stimulation, as well as capable of detecting heart failure, evaluating its severity, tracking the progression thereof, and controlling the delivery of therapy and warnings in response thereto. As explained below in more detail, the IMD 10 may be controlled to monitor cardiac signals and based thereof, to identify potentially abnormal physiology (e.g. ischemia).

The IMD 10 includes a housing 12 that is joined to a header assembly 14 (e.g., an IS-4 connector assembly) that holds receptacle connectors 16, 18, and 20 that are connected to a right ventricular lead 22, a right atrial lead 24, and a coronary sinus lead 26, respectively. The leads 22, 24, and 26 may be located at various locations, such as an atrium, a ventricle, or both to measure the physiological condition of the heart 11. One or more of the leads 22, 24, and 26 detect intra-cardiac electrogram (IEGM) signals that form an electrical activity indicator of myocardial function over multiple cardiac cycles. To sense atrial cardiac signals and to provide right atrial chamber stimulation therapy, the right atrial lead 24 having at least an atrial tip electrode 28, which is typically implanted in the right atrial appendage, and an atrial ring electrode 30. The IEGM signals represent analog cardiac signals that are subsequently digitized and analyzed to identify waveforms of interest. Examples of waveforms identified from the IEGM signals include the P-wave, T-wave, the R-wave, the QRS complex, ST segment and the like.

The coronary sinus lead 26 receives atrial and ventricular cardiac signals and delivers pacing therapy using one or more of a left ventricular tip electrode 32, a left atrial ring electrode 34, and a left atrial coil electrode 36. The right ventricular lead 22 has a right ventricular tip electrode 38, a right ventricular ring electrode 40, a right ventricular (RV) coil electrode 42, and a SVC coil electrode 44. Therefore, the right ventricular lead 22 is capable of receiving cardiac signals, and delivering stimulation in the form of pacing and shock therapy to the right ventricle.

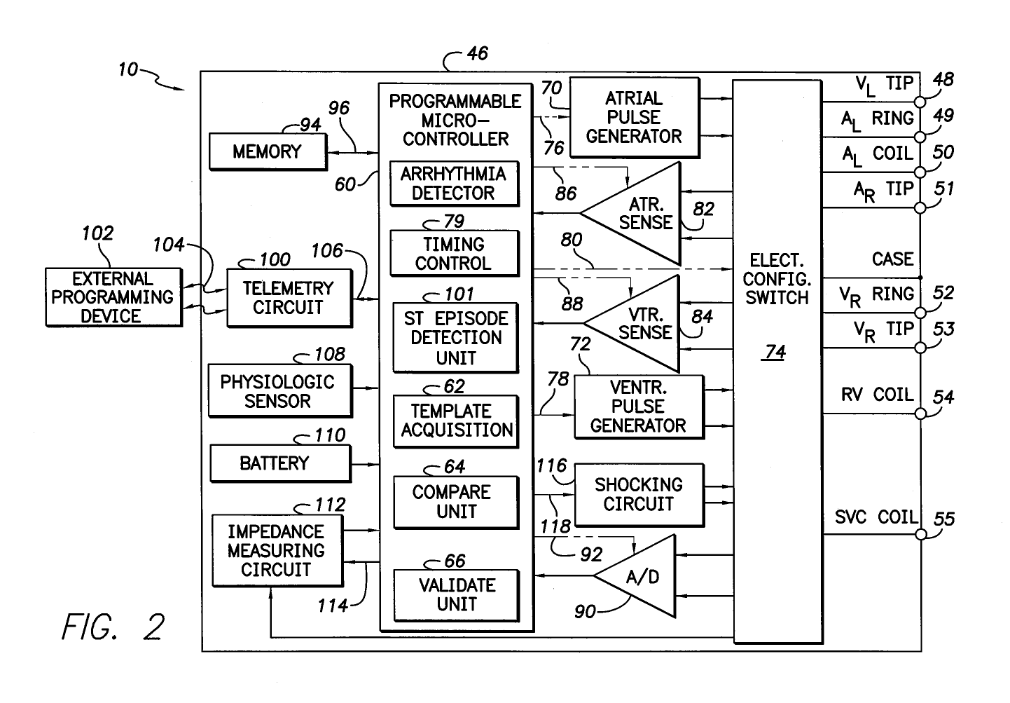

FIG. 2A illustrates a block diagram of exemplary internal components of the IMD 10. The IMD 10 is for illustration purposes only, and it is understood that the circuitry could be duplicated, eliminated or disabled in any desired combination to provide a device capable of treating the appropriate chamber(s) of the heart with cardioversion, defibrillation and/or pacing stimulation.

The housing 46 for IMD 10, is often referred to as the “can”, “case” or “case electrode” and may be programmably selected to act as the return electrode for all “unipolar” modes. The housing 46 further includes a connector (not shown) having a plurality of terminals, namely a right atrial tip terminal (A.sub.R TIP) 51, a left ventricular tip terminal (V.sub.L TIP) 48, a left atrial ring terminal (A.sub.L RING) 49, a left atrial shocking terminal (A.sub.L COIL) 50, a right ventricular tip terminal (V.sub.R TIP) 53, a right ventricular ring terminal (V.sub.R RING) 52, a right ventricular shocking terminal (RV COIL) 54, and an SVC shocking terminal (SVC COIL) 55.

The IMD 10 includes a programmable microcontroller 60, which controls the operation of the IMD 10 based on acquired cardiac signals. For example, the microcontroller 60 may monitor the cardiac signals to identify ST segment shifts and determine ST episodes. The microcontroller 60 (also referred to herein as a processor unit or unit) typically includes a microprocessor, or equivalent control circuitry, is designed specifically for controlling the delivery of stimulation therapy and may further include RAM or ROM memory, logic and timing circuitry, state machine circuitry, and I/O circuitry. Typically, the microcontroller 60 includes the ability to process or monitor input signals (e.g., data) as controlled by a program code stored in memory. Among other things, the microcontroller 60 receives, processes, and manages storage of digitized data from the various electrodes. The microcontroller 60 may also analyze the data, for example, in connection with collecting, over a period of time, reference (baseline and abnormal) morphology templates from the cardiac signals (e.g., sense signals received from leads 22, 24, and 26). As explained below, the microcontroller 60 measure ST segment shifts and compares them to an ST threshold to identify ST episodes and a potential abnormal physiology (e.g., such as when the patient is having a post-myocardial infarct, a “silent” myocardial infarct, a myocardial infarct, an ischemia, a heart block, an arrhythmia, fibrillation, congestive heart failure, an acute myocardial infarction, and the like).

The IMD 10 includes an atrial pulse generator 70 and a ventricular/impedance pulse generator 72 to generate pacing stimulation pulses. In order to provide stimulation therapy in each of the four chambers of the heart, the atrial and ventricular pulse generators, 70 and 72, may include dedicated, independent pulse generators, multiplexed pulse generators, or shared pulse generators. The pulse generators, 70 and 72, are controlled by the microcontroller 60 via appropriate control signals, 76 and 78, respectively, to trigger or inhibit the stimulation pulses.

Switch 74 includes a plurality of switches for connecting the desired electrodes to the appropriate I/O circuits, thereby providing complete electrode programmability. Atrial sensing circuits 82 and ventricular sensing circuits 84 may also be selectively coupled to the leads 22, 24, and 26 through the switch 74 for detecting the presence of cardiac activity in each of the four chambers of the heart. Control signals 86 and 88 from processor 60 direct output of the atrial and ventricular sensing circuits, 82 and 84, that are connected to the microcontroller 60. In this manner, the atrial and ventricular sensing circuits, 82 and 84, are able to trigger or inhibit the atrial and ventricular pulse generators, 70 and 72.

The cardiac signals are applied to the inputs of an analog-to-digital (ND) data acquisition system 90. The data acquisition system 90 is configured to acquire IEGM signals, convert the raw analog data into a digital IEGM signals, and store the digital IEGM signals in memory 94 for later processing and/or telemetric transmission to an external device 102. Control signal 92 from processor 60 determines when the ND 90 acquires signals, stores them in memory 94, or transmits data to an external device 102. The ND 90 is coupled to the right atrial lead 24, the coronary sinus lead 26, and the right ventricular lead 22 through the switch 74 to sample cardiac signals across any combination of desired electrodes.

The microcontroller 60 is coupled to the memory 94 by a suitable data/address bus 96, wherein the programmable operating parameters used by the microcontroller 60 are stored and modified, as required, in order to customize the operation of IMD 10 to suit the needs of a particular patient. The memory 94 may store data indicative of myocardial function, such as the IEGM data, ST segment shifts, reference ST segment shifts, ST segment shift thresholds, trend information associated with ischemic episodes, and the like for a desired period of time (e.g., 6 hours, 12 hours, 18 hours or 24 hours, and the like). The memory 94 may store instructions to direct the microcontroller 60 to analyze the data associated with a plurality of the ischemic episodes by utilizing a termination time at which each of the acute coronary episodes ended and the duration of each of the coronary episodes and/or to identify events of interest, such as an AMI. For example, the memory 94 may store data for each time a shift of the ST segment is detected that exceeds a predetermined threshold.

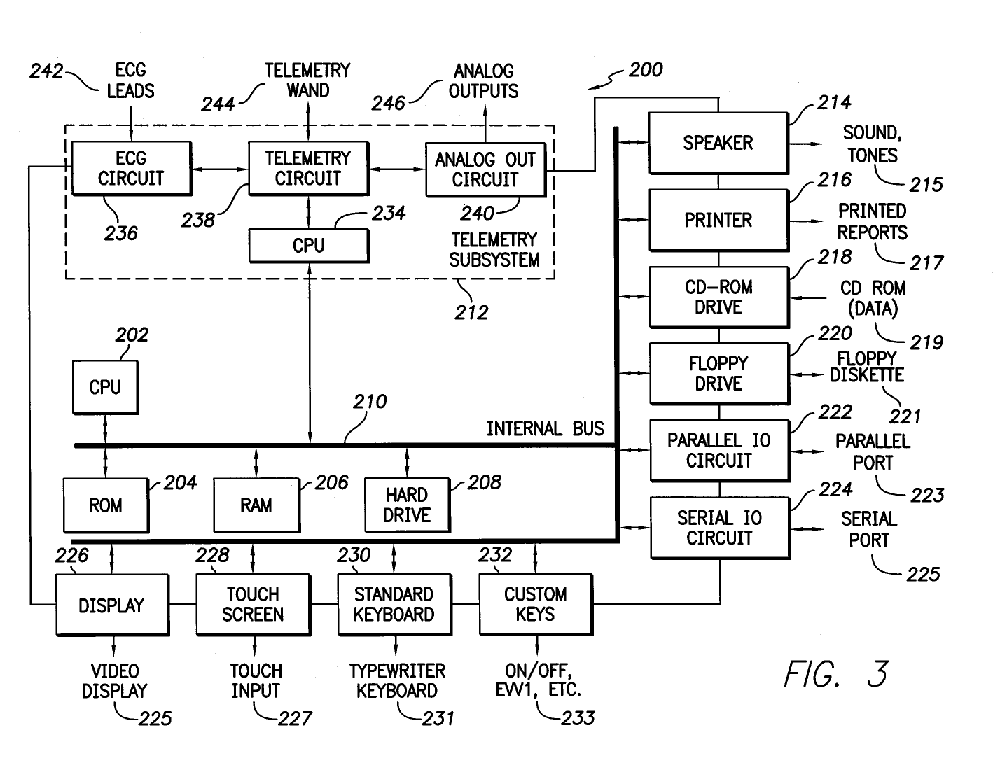

The operating parameters of the IMD 10 may be non-invasively programmed into the memory 94 through a telemetry circuit 100 in communication with the external device 102, such as a programmer (shown in FIG. 3), a trans-telephonic transceiver or a diagnostic system analyzer. The telemetry circuit 100 is activated by the microcontroller 60 by a control signal 106. The telemetry circuit 100 allows intra-cardiac electrograms, and status information relating to the operation of IMD 10 (as contained in the microcontroller 60 or memory 94), to be sent to the external device 102 through an established communication link 104.

The IMD 10 additionally includes a battery 110, which provides operating power to all of the circuits shown within the housing 46, including the processor 60. The IMD 10 is shown as having an impedance measuring circuit 112 which is enabled by the microcontroller 60 via a control signal 114. The impedance measuring circuit 112 is advantageously coupled to the switch 74 so that impedance at any desired electrode may be obtained. The microcontroller 60 controls a shocking circuit 116 by way of a control signal 118. The shocking circuit 116 generates shocking pulses of low (up to 0.5 joules), moderate (0.5-10 joules) or high energy (11 to 40 joules). Such shocking pulses are applied to the heart 11 of the patient through at least two shocking electrodes, and as shown in this embodiment, selected from the left atrial coil electrode 36, the RV coil electrode 42, and/or the SVC coil electrode 44.

The IMD 10 includes an input configured to receive cardiac signals sensed from a heart. The ST episode detection unit 101 is configured to monitor the cardiac signals and identify a potential ST segment shift based thereon.

The microcontroller 60 includes a template acquisition unit 62 that is configured to obtain a reference morphology template based on at least one baseline cardiac signal associated with a normal physiology waveform. Optionally, the template acquisition unit 62 may obtain multiple reference morphology templates. A first reference morphology template may be based on at least one baseline cardiac signal associated with a normal physiology waveform. A second reference morphology template may be based on at least one baseline cardiac signal associated with an abnormal physiology waveform. Optionally, the template acquisition unit 62 may collect a RMT based on sampling of multiple baseline cardiac signals associated with normal physiology waveforms. The RMT may represent an average, mean, mode, median or other statistical combination of features of interest (e.g., QRS complex, number of R-peaks, amplitude of R-peaks, etc.) from the multiple normal physiology waveforms.

The template acquisition unit 62 may store, as the RMT, a digital representation of all or a portion of the waveform forming the baseline cardiac signal. Alternatively, the template acquisition unit 62 may store, as the RMT, values for waveform characteristics that uniquely define the features of interest from the baseline cardiac signal. For example, the template acquisition unit 62 may store, as the RMT, values for one or more of the following waveform characteristics: wave frequency, an area of each QRS segment, a sequence of R-peaks, a number of R-peaks, an amplitude of R-peaks, and a polarity of the R-peaks.

In accordance with an embodiment, the template acquisition unit 62 may collect multiple reference morphology templates. Each of the RMTs is associated with a unique corresponding heart rate zone. When separate RMTs are used for each HR zone, the identifying, comparing and declaring operations utilize the one of the RMTs that corresponds to a present heart rate associated with the cardiac signals sensed.

The microcontroller 60 includes a comparison unit 64 that is configured to compare the cardiac signals to the reference morphology template to derive a morphology indicator representing a degree to which the cardiac signals match the reference morphology template. Optionally, the comparison unit 64 may compare the cardiac signals to first and second reference morphology (RMT) templates to derive first and second morphology indicators, respectively. For example, the first RMT may correspond to a baseline QRS complex with the patient at rest. The second RMT may correspond to an abnormal QRS complex when the patient has an elevated heart rate. The comparison unit 64 may compare one or more of the following waveform characteristics: wave frequency, an area of each QRS segment, a sequence of R-peaks, a number of R-peaks, an amplitude of R-peaks, and a polarity of the R-peaks. Optionally, the comparison unit 64 may identify a polarity of a dominant R-peak in the cardiac signal, and compare the polarity of the dominant R-peak in the cardiac signal with a polarity of a dominant R-peak in the reference morphology template, the validation unit configured to validate the actual ST segment shift based in part on the polarity comparison.

The microcontroller 60 includes a validation unit 66 that is configured to declare the potential ST segment shift to be an actual ST segment shift based on the morphology indicator. When multiple RMT are used for comparison, the validation unit 66 considers the associated corresponding multiple morphology indicators before declaring the potential ST segment shift to be an actual ST segment shift or a false positive.

The ST episode detection unit 101 declares an ST episode when a predetermined number of actual ST segment shifts are validated. The sensing, identifying, comparing and declaring functions are repeated by the microcontroller for sets of cardiac events. Each of the cardiac event is associated with a heart beat. The ST episode detection unit 101 declares a set of the cardiac events to exhibit ST segment shift when a predetermined number of cardiac events in the corresponding set have morphology indicators indicating a predetermined degree of match with the reference morphology template.

FIG. 3 illustrates a functional block diagram of an external device 200, such as a programmer, that is operated by a physician, a health care worker, or a patient to interface with IMD 10. The external device 200 may be utilized in a hospital setting, a physician's office, or even the patient's home to communicate with the IMD 10 to change a variety of operational parameters regarding the therapy provided by the IMD 10 as well as to select among physiological parameters to be monitored and recorded by the IMD 10. For example, the external device 200 may be used to program coronary episode related parameters, such as ischemia-related and AMI-related ST segment shift thresholds, duration thresholds, and the like. The external device 200 may be used to program one or more of the following waveform characteristics: wave frequency, an area of each QRS segment, a sequence of R-peaks, a number of R-peaks, an amplitude of R-peaks, and a polarity of the R-peaks. Further, the external device 200 may be utilized to interrogate the IMD 10 to determine the condition of a patient, to adjust the physiological parameters monitored or to adapt the therapy to a more efficacious one in a non-invasive manner.

During the template acquisition phase, the external device 200 may be used to verify whether a cardiac signal associated with a heart beat may be processed to form a reference morphology template. For example, the physician may inform the IMD 10, through the external device 200, when a particular heart beat or group of heart beats are normal, and thus the IMD 10 may acquire baseline or normal RMT(s) from such heart beat(s). When abnormal RMTs are used, the physician may inform the IMD 10, through the external device 200, when particular heart beat or group of heart beats are abnormal and thus the IMD 10 may acquire abnormal or reverse RMT(s) from such heart beat(s). Similarly, when multiple HR zones are used, the physician may inform the IMD 10, through the external device 200, when particular heart beat or group of heart beats are associated with a HR zone and thus the IMD 10 may acquire RMT(s) from such heart beat(s) associated with the corresponding HR zones.

External device 200 includes an internal bus 210 that connects/interfaces with a Central Processing Unit (CPU) 202, ROM 204, RAM 206, a hard drive 208, a speaker 214, a printer 216, a CD-ROM drive 218, a floppy drive 220, a parallel I/O circuit 222, a serial I/O circuit 224, a display 226, a touch screen 228, a standard keyboard connection 230, custom keys 232, and a telemetry subsystem 212. The internal bus 210 is an address/data bus that transfers information (e.g., either memory data or a memory address from which data will be either stored or retrieved) between the various components described. The hard drive 208 may store operational programs as well as data, such as reference ST segments, ST thresholds, timing information and the like. The hard drive 208 may store reference and abnormal RMTs, waveform characteristics, values for waveform characteristics, and the like.

The CPU 202 typically includes a microprocessor, a micro-controller, or equivalent control circuitry, designed specifically to control interfacing with the external device 200 and with the IMD 10. The CPU 202 may further include RAM or ROM memory, logic and timing circuitry, state machine circuitry, and I/O circuitry to interface with the IMD 10. Typically, the microcontroller 60 includes the ability to process or monitor input signals (e.g., data) as controlled by program code stored in memory (e.g., ROM 206).

In order for a physician or health care worker to communicate with the external device 200, a display 226, a touch screen 228, a standard keyboard 230, and custom keys 232 are provided. The display 226 (e.g., may be connected to a video display 225) and the touch screen 228 display text, alphanumeric information, data and graphic information via a series of menu choices to be selected by the user relating to the IMD 10, such as for example, status information, operating parameters, therapy parameters, patient status, access settings, software programming version, ST segment thresholds, and the like. The touch screen 228 accepts a user's touch input 227 when selections are made. The keyboard 230 (e.g., a typewriter keyboard 231) allows the user to enter data to the displayed fields, operational parameters, therapy parameters, as well as interface with the telemetry subsystem 212. Furthermore, custom keys 232 turn on/off 233 (e.g., EVVI) the external device 200, a printer 216 prints hard-copies of any reports 217 for a physician/healthcare worker to review or to be placed in a patient file, and speaker 214 provides an audible warning (e.g., sounds and tones 215) to the user in the event a patient has any abnormal physiological condition occur while the external device 200 is being used. In addition, the external device 200 includes a parallel I/O circuit 222 to interface with a parallel port 223, a serial I/O circuit 224 to interface with a serial port 225, a floppy drive 220 to accept floppy diskettes 221, and a CD-ROM drive 218 that accepts CD ROMs 219.

The telemetry subsystem 212 includes a central processing unit (CPU) 234 in electrical communication with a telemetry circuit 238, which communicates with both an ECG circuit 236 and an analog out circuit 240. The ECG circuit 236 is connected to ECG leads 242. The telemetry circuit 238 is connected to a telemetry wand 244. And, the analog out circuit 212 includes communication circuits, such as a transmitting antenna, modulation and demodulation stages (not shown), as well as transmitting and receiving stages (not shown) to communicate with analog outputs 246. The external device 200 may wirelessly communicate with the IMD 10 and utilize protocols, such as Bluetooth, GSM, infrared wireless LANs, HIPERLAN, 3G, satellite, as well as circuit and packet data protocols, and the like. The wireless RF link utilizes a carrier signal that is selected to be safe for physiologic transmission through a human being and is below the frequencies associated with wireless radio frequency transmission. Alternatively, a hard-wired connection may be used to connect the external device 200 to IMD 10 (e.g., an electrical cable having a USB connection).

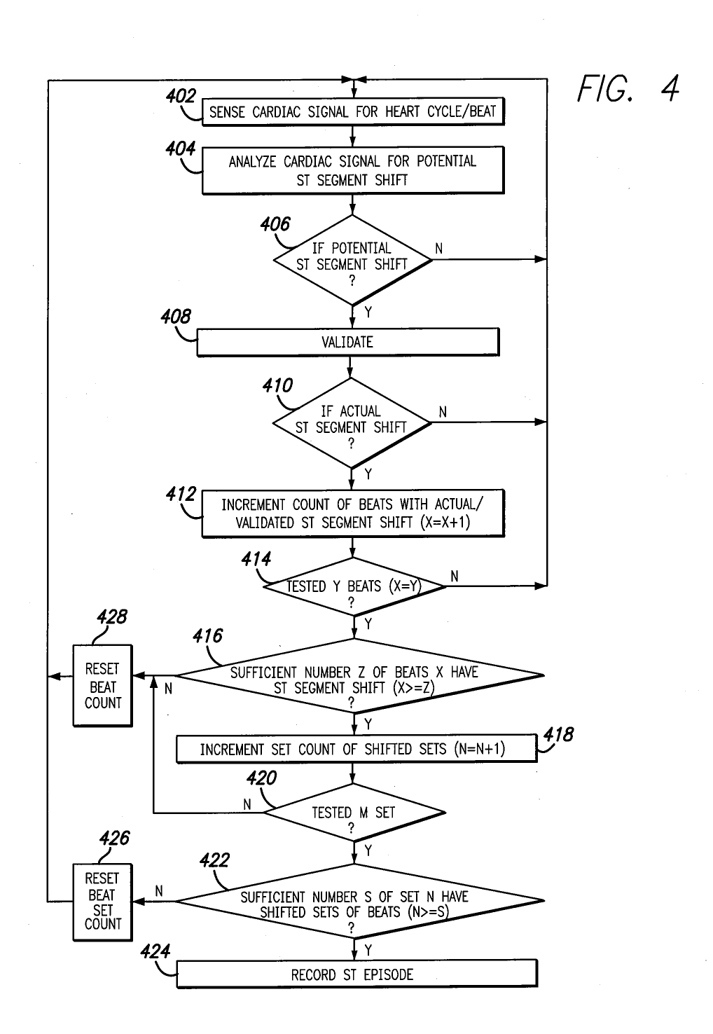

FIG. 4 illustrates an example of an ST-MD process that includes validation based on RMTs in accordance with an embodiment. Beginning at 402, the method senses a cardiac signal for a heart cycle or beat. At 404, the method analyzes the cardiac signal for potential ST segment shift. The analysis at 404 may be performed in accordance with various algorithms. For example, analysis methods are described in U.S. Pat. No. 8,090,435 to Gill et al. and entitled “System and method for distinguishing among cardiac ischemia, hypoglycemia and hyperglycemia, using an implantable medical device”, and in U.S. Pat. No. 8,180,439 to Gill et al. and entitled “Ischemia Detection Using Intra-Cardiac Signals”, both of which are expressly incorporated herein by reference in their entirety.

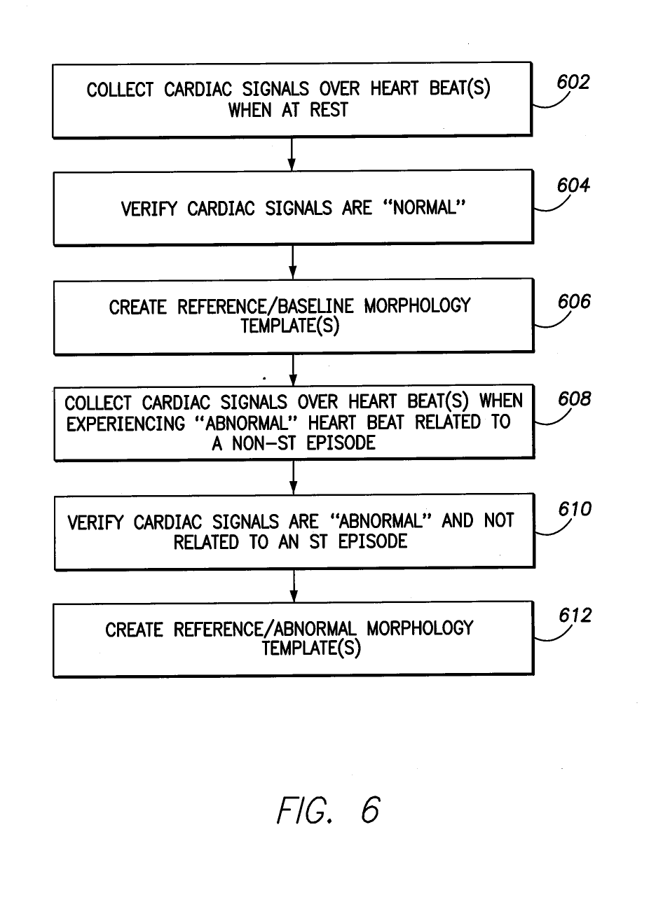

At 406, the method determines whether a potential ST segment shift was identified at 404. If not, flow returns to 402. If so, flow moves to 408. At 408, the potential ST segment shift is “validated” in accordance with various embodiments discussed hereafter. As explained in connection with FIGS. 6-8, the potential ST segment shift is validated based on various RMTs that have been previously collected.

At 410, the method determines whether an actual ST segment shift occurred or whether a false positive declaration (FPD) was determined in the validation process at 408. When a FPD is determined, the beat is “ignored” and not counted in the subsequent operations of FIG. 4. Instead, when an FPD is determined, flow returns to 402 and a new cardiac signal is measures. Alternatively, at 410, when an actual ST segment shift is identified, flow moves to 412.

At 412, the heart beat exhibiting the actual ST segment shift is counted and a running beat count is incremented (X=X+1). At 414, the method determines whether the number of beats tested in a current set of beats has reached a full or complete set (e.g., Y beats). If not, flow returns to 402 and a new heart beat is measures. If so, flow moves to 416.

At 416, the method determines whether a sufficient predetermined number (Z) of beats (X) have been actual ST segment shifts (X>=Z). For example, if Z=6 and Y=8, then the method determines whether 6 out of the last 8 beats experienced actual ST segment shift. The count of 8 beats may include beats that were determined to have FPD at 408. When an insufficient number of beats exhibit validated or actual ST segment shift, flow moves to 428. At 428, the beat counters, X and Y are reset and flow returns to 402. When a sufficient number of beats exhibit validated or actual ST segment shift, flow moves to 418.

At 418, the method increments a set count (N=N+1). The set count tracks the number of sets of beats that include the predetermined number of beats with ST segment shift. At 420, the method determines whether a sufficient predetermined number of sets M have been tested (e.g., 3, 5, etc.). If not, flow returns to 428 where the beat counters, X and Y are reset and flow returns to 402. At 420, when the predetermined number of sets M has been tested, flow moves to 422.

At 422, the method determines whether a sufficient predetermined number (S) of sets represent shifted sets (N>=S). If not, the method determines that the patient is not experiencing an ST episode and flow moves to 426. At 426, the beat and set counters are reset and a new process is started. Optionally, the set counter may not be reset, but instead the oldest set may be removed in order that the process of FIG. 4 continues to look for any successive number S of sets of beats that exhibit shift.

Alternatively at 424, if the method determines that the sufficient number (e.g., S=3) of successive sets of beats exhibit shift, then the method determines that the patient is in fact experiencing an ST episode. At 424, the method records the ST episode and performs various other operations, such as delivering therapy, recording other patient information and the like.

It should be recognized that the example of FIG. 4 is merely one type of ST morphology discrimination process that may be used for identifying ST episodes. There are other ST morphology discrimination processes that may incorporate the ST segment shift validation methods and systems described herein.

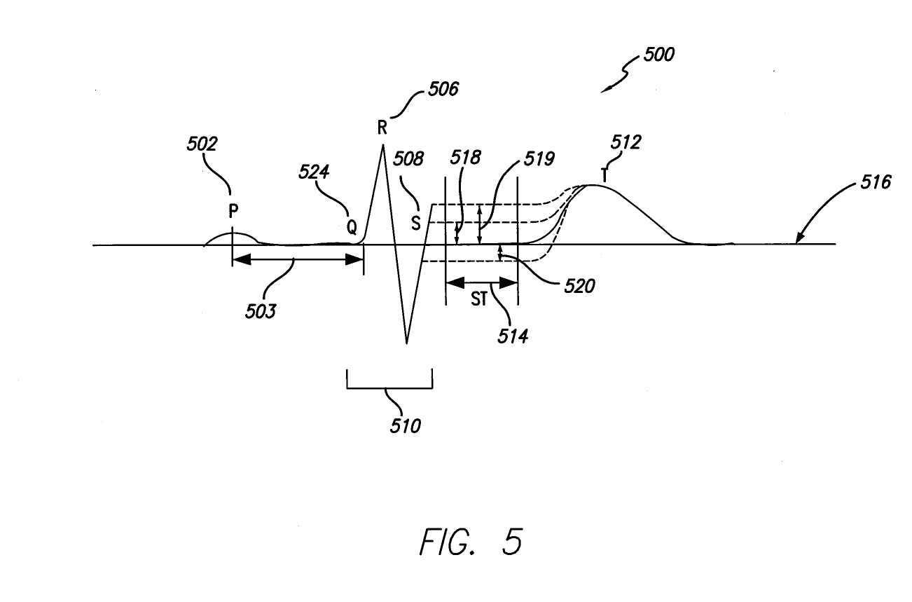

FIG. 5 illustrates a single cardiac cycle 500 composed of a P-wave 502, a Q-wave 504, an R-wave 506, an S-wave 508, and a T-wave 512. The cardiac cycle 500 may represent cardiac signals, such as intra-cardiac electrogram (IEGM) signals, electrocardiogram (ECG) signals, and the like. The horizontal axis represents time, while the vertical axis is defined in units of voltage. An abnormal cardiac signal indicates a potential ischemic condition. A QRS complex 510 is composed of a Q-wave 504, an R-wave 506, and an S-wave 508. The QRS complex 510 is used to locate the R-wave 506 to determine a baseline 516. The portion of the signal between the S-wave 508 and T-wave 512 constitutes a ST segment 514. As shown, the ST segment 514 may have a voltage level that aligns with the voltage level of the baseline 516. Alternatively, the ST segment 514 may have a voltage level that is shifted above 518, 519 or shifted below 520 the baseline 516. Therefore, ST segment variations 518-520 may occur above or below the baseline 516.

As used throughout, the term ST segment variations 518-520 is used to include ST segment deviations or ST segment shifts. An ST segment deviation is determined by subtracting the level of a PQ segment 503 from the level of the ST segment 514 for one heartbeat. The ST segment deviation provides a measure of the change in variability over a period of time. An ST segment shift is determined by variations in the ST segment deviation over a period of time. For example, a current ST segment shift maybe calculated by subtracting a stored baseline ST segment deviation from a newly acquired ST segment deviation. ST segment deviations and ST segment shifts maybe calculated as averages over multiple cardiac cycles as well. Deviations of the voltage level of the ST segment 514 may be a result of injury to cardiac muscle, variations in the synchronization of ventricular muscle depolarization, drug or electrolyte influences, and the like. The voltage elevation of the ST segment 514, as shown by 518 and 519, in a cardiac signal may result when there are abnormalities in the polarizations of cardiac tissue during an acute myocardial infraction (AMI). The STS variations 518-520 may arise because of differences in the electrical potential between cells that have become ischemic and those that are still receiving normal blood flow. Thus, the ST segment variations 518-520 are a reliable indicator of the possibility of ischemia. It is recognized that ST segment 514 may deviate due to non-ischemic events. The exemplary embodiments set forth above, and hereafter, may be presented in connection with ST segment shifts and/or ST segment deviations. It is understood that, in accordance with an alternative embodiment, the systems and methods described herein may be implemented utilizing of the ST segment deviation or ST segment shift, throughout collectively referred to as ST segment variations 518-520.

A shift in the ST segment 514 can be caused by non-ischemia related factors, such as “axis shifts”, electrical noise, cardiac pacing, high sinus or tachycardia cardiac rates that distort the IEGM waveform. The measured ST segments 514 may include noise. Upon extracting the ST segment 514 from the noise it is possible to discriminate the occurrence of an ischemia. There may be a shift in the ST segment 514 that does not indicate an ischemic condition. As explained below in more detail, in accordance with certain embodiments of the present invention, shifts in the ST segment that are due to ischemia related events can be discriminated from shifts in the ST segment that are due to non-ischemia related events. The discrimination of ischemia related and non-ischemia related shifts in the ST segment are achieved through a statistical determination of the variability of the ST segment shift. The ST segment shifts are collected to obtain a ST threshold. The ST threshold is used in a comparison with subsequently measured ST segment shifts to identify potentially abnormal physiology.

FIG. 6 illustrates a process for acquiring active “Normal” (baseline) and “Abnormal” (reverse) reference morphology templates (RMTs). Beginning at 602, the method collects cardiac signals associated with one or more heart beat(s) while the patient is at rest.

At 604, the method verifies that the cardiac signals are “normal”. For example, an IMD or external programmer may automatically analyze the cardiac signals utilizing various techniques to verify that the heart beat represents a normal heart beat. Optionally, a physician may analyze the cardiac signals, such as by reviewing IEGM or ECG waveforms and other data collected from the patient, to verify that the heart beat represents a normal heart beat.

Once the method confirms that a normal heart beat occurred, the method creates reference/baseline morphology template associated with the heart beat. The template may represent an IEGM waveform corresponding to the cardiac signal. Alternatively, the template may represent values for one or more parameters that are measured from an IEGM waveform, such as a wave frequency, an area of each QRS segment, a sequence of R-peaks, a number of R-peaks, the amplitude of R-peaks, a polarity of the R-peaks and the like. In accordance with the above process, one reference morphology template is generated based on one or more heart beats. In the foregoing example, each template may be created based on a cardiac signal associated with a single heart beat. Optionally, each template may be created based on cardiac signals associated with an ensemble of multiple heart beats, where the template is created from an average, mean, median, mode or other statistical parameters associated with the ensemble of heart beats.

Optionally, the method may create multiple reference morphology templates. For example, the process at 602-606 may be repeated multiple times in connection with heart beats in different heart rate (HR) zones (e.g., less than 60, 60-80, 80-120, 120-140, 140-160, greater than 160). The process at 602-606 may create one or more reference morphology templates associated with each HR zone.

Next at 608, the method collects cardiac signals associated with one or more heart beat(s) while the patient is experiencing an abnormal heart beat related to a non-ST episode. For example, the abnormal heart beat may occur while experiencing rate dependent bundle branch blocks, posture-related axis changes of the EGM signal and other non-ST segment related physiologic behavior.

At 610, the method verifies that the cardiac signals are “abnormal”. For example, an IMD or external programmer may automatically analyze the cardiac signals utilizing various techniques to verify that the heart beat represents an abnormal heart beat. Optionally, a physician may analyze the cardiac signals, such as by reviewing IEGM or ECG waveforms and other data collected from the patient, to verify that the heart beat represents an abnormal heart beat (e.g. rate dependent bundle branch blocks, posture-related axis changes of the EGM signal and other non-ST segment related physiologic behavior).

Once the method confirms that an abnormal heart beat occurred, the method creates reference morphology template associated with the heart beat. The template may represent an IEGM waveform corresponding to the cardiac signal. Alternatively, the template may represent values for one or more parameters that are measured from an IEGM waveform, such as a wave frequency, an area of each QRS segment, a sequence of R-peaks, a number of R-peaks, the amplitude of R-peaks, a polarity of the R-peaks and the like. In accordance with the above process, one reference morphology template is generated based on one or more heart beats. Optionally, each abnormal morphology template may be created based on cardiac signals associated with an ensemble of multiple heart beats, where the template is created from statistical parameters associated with the ensemble of heart beats.

In accordance with the foregoing, RMTs are created and stored. The RMTs may be created by the IMD 10 or by an external device 200 and then uploaded to the IMD 10. The RMTs are then used during validation to seek to avoid FPDs.

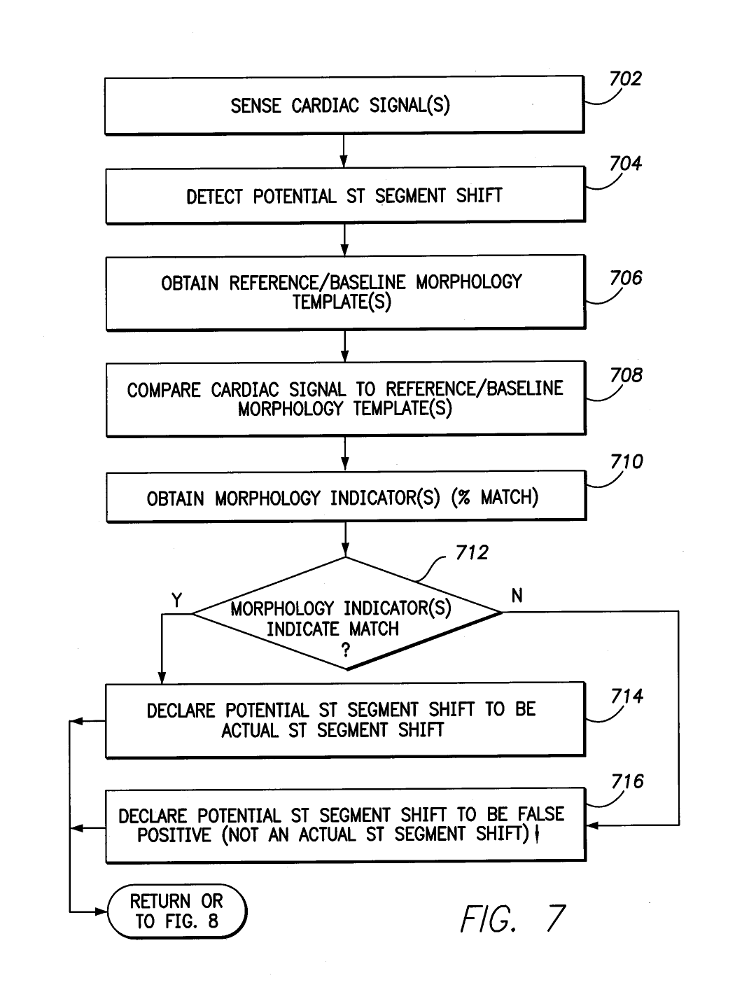

FIG. 7 illustrates a processing sequence carried out to perform a cross-check validation for potential ST segment shifts that are detected by an ST-MD monitoring process. For example, the process of FIG. 7 may be performed during the validation operation at 408 in FIG. 4. In FIG. 7, the operations at 702 and 704 correspond to the operations at 402 and 404 in FIG. 4. At 702, the method senses cardiac signal(s) and at 704, the method determines whether a potential ST segment shift is detected. Hence, when the process of FIG. 7 is used in combination with the process of FIG. 4, the operations at 702 and 704 are removed (as they are already performed at 402 and 404). Alternatively, when the process of FIG. 7 is used independent of, and without the process of FIG. 4, then the operations at 702 and 704 are implemented.

At 706, the method obtains, from a template storage (e.g., in the IMD or external device), one or more reference/baseline morphology template(s). As one example, a single template may be used for all cardiac signals. Alternatively, a different template may be used based on the HR zone associated with the cardiac signal. Optionally, different templates may be chosen based on additional factors, such as the time of day, various physiologic characteristics exhibited by the patient and the like.

At 708, the method compares the cardiac signal(s) to one or more reference/baseline morphology template(s). The comparison may include various operations. For example, the comparison may include comparing a shape of the QRS complex in the measured cardiac signal with the QRS complex in the template (e.g., such as using a least mean squares comparison and the like). Optionally, the comparing operation may include comparing values for one or more of the following waveform parameters: wave frequency, an area of each QRS segment, a sequence of R-peaks, a number of R-peaks, an amplitude of R-peaks, and a polarity of the R-peaks. For example, the method may analyze the measured cardiac signal and derive values for the waveform parameters of interest. The values associated with the measured cardiac signals are then compared to values associated with the template for the waveform parameters.

At 710, the method obtains one or more morphology indicator(s) based on the comparisons at 708. The morphology indicator may represent an indication of a degree to which an individual comparison represents a match. For example, the comparison may indicate a high, medium or low degree of correlation between the measured cardiac signal and the template. Optionally, the comparison may indicate a percentage (%) to which the measured cardiac signal and the template match. For example, when the waveform parameter represents the number of R-peaks, the comparison may determine that 90% of the R-peaks in the measured cardiac signal match the R-peaks in the template. Hence, the morphology indicator would be afforded a value of 90%.

Optionally, more than one waveform parameter may be used with each waveform parameter being weighted, either equally or by different amounts based on importance. For example, when the waveform parameters represent a sequence of R-peaks and an area of the QRS segment, the comparison may determine that 70% of the sequence of R-peaks in measured cardiac signal match the sequence of R-peaks in the template, while 90% of the area of the QRS segment in the measured cardiac signal match the area of the QRS segment in the template. If each waveform parameter is weighted equally, then the method may determine that the morphology indicator is 80% ([70+90]/2).

At 712, the method determines whether the morphology indicator(s) (MI) determined at 710 indicate that a match exists. The test at 712 may represent a simple comparison of a percentage to a predetermined or preprogrammed MI match threshold. For example, the MI match threshold may be set to 85% or greater. Hence, in the above examples, when the MI equals 80% this would indicate that a match does not exist. When the MI equals 90% this would indicate that a match does exist. Alternatively, when high, medium, low values are used for the MI, the test at 712 may be programmed to require a “high” degree of correlation to constitute a match. Based on the test at 712, flow branches to 714 or 716.

When a match exists, flow moves to 714. At 714, the method declares the potential ST segment shift to be an actual ST segment shift. When a match does not exist, flow moves to 716. At 716, the method declares the potential ST segment shift to be a FPD and not an actual ST segment shift.

In accordance with an embodiment, the method returns from 714 or 716 to the ST-MD process. If the potential ST segment shift is validated, namely declared to be an actual ST segment shift, then the ST-MD process uses the measured cardiac signal to update the ST segment monitoring information (e.g., a running count of beats or sets that exhibit ST segment shift). Alternatively, if the potential ST segment shift is not validated, namely declared to be a FPD and not an actual ST segment shift, then the ST-MD process ignores or disregards the measured cardiac signal and does not use the measured cardiac signal to update the ST segment monitoring information.

Optionally, in accordance with another embodiment, a second cross check may be utilized to determine whether the potential ST segment shift is an actual ST segment shift or a false positive. The second cross check may be based on abnormal RMTs.

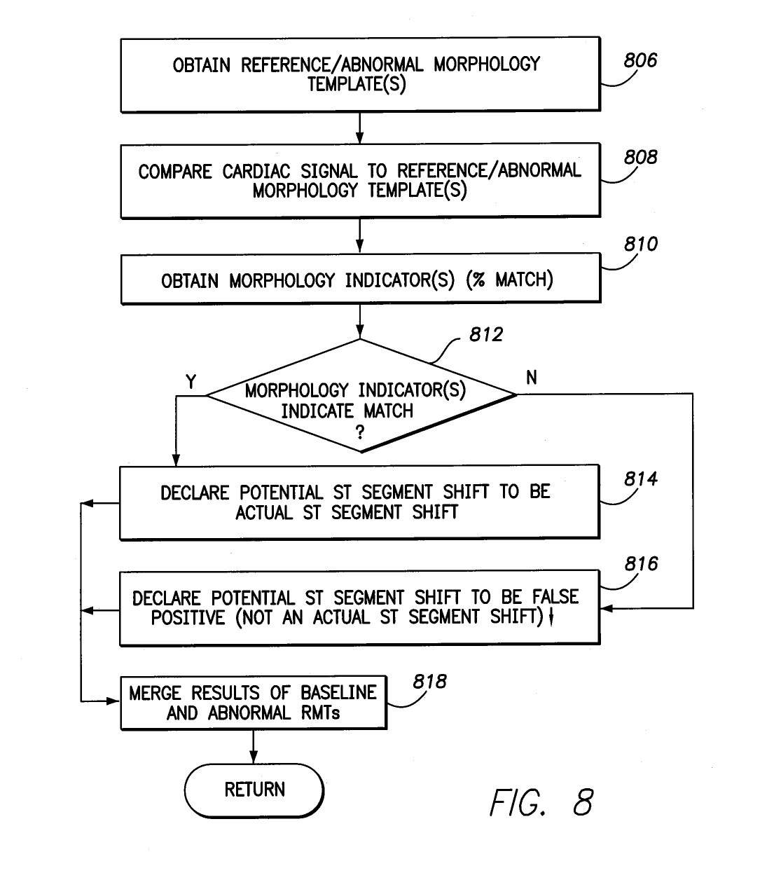

FIG. 8 illustrates a processing sequence carried out to perform a cross-check validation for potential ST segment shifts that are detected by an ST-MD. In FIG. 8, flow begins after completion of the method in FIG. 7, and thus the measured cardiac signal shall refer to the cardiac signal(s) sensed in FIG. 7. The potential ST segment shift shall represent the potential ST segment shift detected in FIG. 7 (or FIG. 4).

At 806, the method obtains, from template storage, one or more reference/abnormal morphology template(s). As one example, a single template may be used for all cardiac signals. Alternatively, a different template may be used based on the HR zone associated with the cardiac signal. Optionally, different templates may be chosen based on additional factors, such as the time of day, various physiologic characteristics exhibited by the patient and the like.

At 808, the method compares the cardiac signal(s) to one or more reference/abnormal morphology template(s). The comparison may include various operations at explained above in connection with FIG. 7. At 810, the method obtains one or more morphology indicator(s) based on the comparisons at 808. As explained in connection with FIG. 7, the morphology indicator may represent an indication of a degree to which an individual comparison represents a match (e.g., high, medium or low degree of correlation, or percentage (%)). As in FIG. 7, more than one waveform parameter may be used with each waveform parameter being weighted, either equally or by different amounts based on importance.

At 812, the method determines whether the morphology indicator(s) (MI) determined at 810 indicate that a match exists. The test at 812 may represent a simple comparison of a percentage to a predetermined or preprogrammed MI match threshold. Alternatively, when high, medium, low values are used for the MI, the test at 812 may be programmed to require a “high” degree of correlation to constitute a match. Based on the test at 812, flow branches to 814 or 816.

When a match exists, flow moves to 814. At 814, the method declares the potential ST segment shift to be an actual ST segment shift. When a match does not exist, flow moves to 816. At 816, the method declares the potential ST segment shift to be to be a FPD and not an actual ST segment shift.

At 818, the method merges the analysis from the tests at 812 (relative to an abnormal morphology template) and 712 (relative to a baseline morphology template) to determine whether to inform the ST-MD process that the measured cardiac signal represents an actual ST segment shift or a false positive. The analysis at 818 may be programmed. For example, if either of the tests at 712 and 812 indicates a false positive, then the method may inform the ST-MD process that the measured cardiac signal represents a false positive. Optionally, if both of the tests at 712 and 812 indicate an actual ST segment shift, then the method may inform the ST-MD process that the measured cardiac signal represents an actual ST segment shift. Alternatively, the analysis at 818 may consider the values of the morphology indicators. For example, if the MI determined at 710 indicates a 80% probability of a match between the measured cardiac signal and the baseline morphology template, while the MI determined at 810 indicates a 50% probability of a match between the measured cardiac signal and the abnormal morphology template, then the method may inform the ST-MD process that the measured cardiac signal represents an actual ST segment shift.

If the potential ST segment shift is validated, namely declared to be an actual ST segment shift, then the ST-MD process uses the measured cardiac signal to update the ST segment monitoring information. Alternatively, if the potential ST segment shift if not validated, namely declared to be a false positive and not an actual ST segment shift, then the ST-MD process ignores or disregards the measured cardiac signal and does not use the measured cardiac signal to update the ST segment monitoring information. Flow moves from 818 to return to the ST-MD process that originally designated a measured heart beat to exhibit ST segment shift.

In accordance with the methods and systems described herein reference morphology templates are uses as morphology-based filters to reduce false or inappropriate ST episode detections whenever an ST shift episode is otherwise diagnosed in an ST-MD process.

ST monitoring uses a unipolar (can to RV tip) signal from the right ventricular intracardiac high voltage lead. Prior research, regarding morphology based algorithms to discriminate VT from SVT, have demonstrated the RV-can vector to be superior to SVC-Can as the EGM source. Therefore, in accordance with embodiments of the present invention, the morphology-based filter may be applied over the unipolar ST channel. Optionally, the morphology-based filter may be applied over other sensing channels or vectors.

Further, while certain systems herein are described in the context of an IMD located outside of the heart and coupled to one or more leads in the heart, optionally the above described methods may be implemented in connection with leadless implantable devices that are entirely located within one or more chambers of the heart.

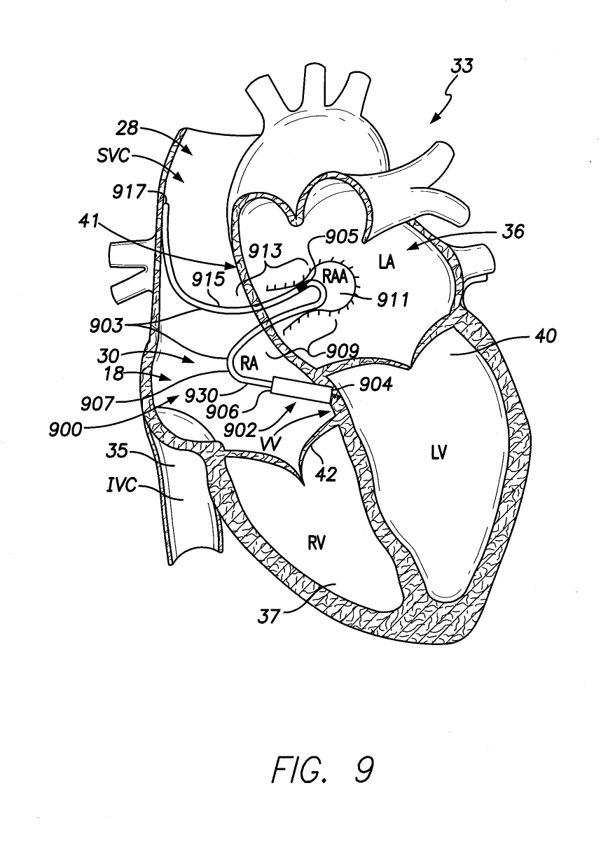

FIG. 9 provides a sectional view of a patient's heart 33 and shows a leadless implantable medical device (LIMD) 900 that may implement the methods of FIGS. 4-8. The LIMD 900 comprises a housing 902 configured to be implanted entirely within a single local chamber of the heart. The housing 902 includes a proximal base end 904 and a distal top end 906. The proximal base end 904 includes an active fixation member, such as a helix, that is illustrated to be implanted in the ventricular vestibule (VV). A shaped intra-cardiac (IC) device extension 903 may be provided that extends from the distal top end 906 of the housing 902. The IC device extension 903 comprises an elongated body that may be tubular in shape and may include a metal braid provided along at least a portion of the length therein (as explained herein in more detail). The extension body including a transition sub-segment, an active interim-segment and a stabilizer end-segment, all of which are illustrated in a deployed configuration and some of which are preloaded against anatomical portions of tissue of interest. For example, the active interim-segment (e.g., second curved segment 911, and all or portions of the first and second linear regions 909 and 913) and the stabilizer end-segment (e.g., third curved segment 915 and all or portions of the second linear region 913) are shown preloaded against anatomical tissue of interest. The braid resists torque compression but permits lateral flex. One or more electrodes 905 are carried by the IC device extension 903 and are electrically connected to electronics within the housing 902 through conductors extending through the body of the IC device extension.

The IC device extension 903 includes a short stem 930 that extends a short distance from the distal top end 906 of the housing 902. The stem 930 merges into a first curved segment 907 that turns at a sharp angle with respect to a longitudinal axis of the housing 902. The first curved segment 907 merges into and is followed by a first generally linear region 909 that extends laterally from the housing 902, along a lateral axis, until merging with a second curved segment 911. The second curved segment 911 turns at a sharp angle with respect to the longitudinal axis of the housing 902 and the lateral axis of the first linear region 909. As one example, the second curved segment 911 may approximate a 180 degree sharp or “hairpin” curve away from the lateral axis of the first linear region 909 and away from the longitudinal axis of the housing 902. The second curved segment 911 merges into and is followed by a second generally linear region 913 that extends along a second lateral direction.

One or more electrodes 905 are located along the second curved segment 911. Optionally, the electrode(s) may be provided in the region proximate to the junction of the second curved segment 911 and the second linear region 913. Optionally, one or more electrodes 905 may be provided along the second linear region 913.

The second linear region 913 merges with and extends to a third curved segment 915. The third curved segment 915 follows an extending “slow” arc and then terminates at a tail end 917 of the IC device extension 903. The third curved segment 915 follows a slow arc with respect to the longitudinal axis of the housing 902 and the lateral axis of the first linear region 909. The LIMD 900 is configured to place the housing 902 in the lower region of the right atrium between the OS and IVC with a distal helix electrode, on the housing 902, in the ventricular vestibule to provide ventricular pacing and sensing. The IC device extension 903 extends upward in the right atrium toward and into the SVC. The IC device extension 903 is configured (length wise and shape wise) such that the second curved segment 911 may be implanted within the right atrial IC device extension (RAA), along with those portions of the first and second linear regions 909, 913 near the second curved segment 911. The configuration in FIG. 9 places the electrode 905 in the RAA to allow for right atrial pacing and sensing. The configuration in FIG. 9 also places the proximal portion of the third curved segment 915 against a wall of the SVC to provide overall stability to the LIMD 900.

Optionally, the IC device extension 903 may be omitted entirely. Optionally, the LIMD 900 may be implanted in another chamber, such as in the RV, LV, and/or LA. Optionally, other types of extensions may be provided that extend from the LIMD housing 902. Optionally, multiple LIMD may be implanted into the heart, such as with one LIMD in the RA and another LIMD in the RV, or one LIMD in the LV and another LIMD in the RV. The methods described in connection with FIGS. 4-8 may be implemented with any of the above discussed LIMD as well as other types of LIMD not specifically described herein.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions, types of materials and coatings described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. §112, sixth paragraph, unless and until such claim limitations expressly use the phrase “means for” followed by a statement of function void of further structure.

What is claimed is:

1. A method for ST morphology discrimination, comprising:
sensing cardiac signals of a heart;
obtaining a reference morphology template based on at least one baseline cardiac signal associated with a normal physiology waveform;
identifying a potential ST segment shift from the cardiac signals;
comparing the cardiac signals to the reference morphology template to derive a morphology indicator representing a degree to which the cardiac signals match the reference morphology template; and
declaring the potential ST segment shift to be an actual ST segment shift based on the morphology indicator.

2. The method of claim 1, wherein the obtaining operation includes obtaining first and second reference morphology templates, the first reference morphology template based on at least one baseline cardiac signal associated with a normal physiology waveform, the second reference morphology template based on at least one baseline cardiac signal associated with an abnormal physiology waveform.

3. The method of claim 2, wherein the comparing operation compares the cardiac signals to the first and second reference morphology templates to derive first and second morphology indicators, respectively.

4. The method of claim 3, wherein the declaring operation declares the potential ST segment shift to be an actual ST segment shift based on the first and second morphology indicators.

5. The method of claim 1, further comprising collecting a reference morphology template based on sampling of multiple baseline cardiac signals associated with normal physiology waveforms.

6. The method of claim 1, wherein the sensing, identifying, comparing and declaring operations are repeated for sets of cardiac events, each cardiac event associated with a heart beat, the method further comprising declaring a set of the cardiac events to exhibit ST segment shift when a predetermined number of cardiac events in the corresponding set have morphology indicators indicating a predetermined degree of match with the reference morphology template.

7. The method of claim 1, further comprising collecting multiple reference morphology templates, each of which is associated with a unique corresponding heart rate zone, the identifying, comparing and declaring operations utilizing one of the multiple reference templates corresponding to a present heart rate associated with the cardiac signals sensed.

8. The method of claim 1, further comprising identifying a polarity of a dominant R-peak in the cardiac signal, comparing the polarity of the dominant R-peak in the cardiac signal with a polarity of a dominant R-peak in the reference morphology template, the declaring operation based in part on the polarity comparison.

9. The method of claim 1, wherein the comparing operation includes comparing one or more of the following: wave frequency, an area of each QRS segment, a sequence of R-peaks, a number of R-peaks, an amplitude of R-peaks, and a polarity of the R-peaks.

10. A system, comprising:
an input configured to receive cardiac signals sensed from a heart;
an ST episode detection unit configured to monitor the cardiac signals and identify a potential ST segment shift based thereon;
a template acquisition unit configured to obtain a reference morphology template based on at least one baseline cardiac signal associated with a normal physiology waveform;
a comparison unit configured to compare the cardiac signals to the reference morphology template to derive a morphology indicator representing a degree to which the cardiac signals match the reference morphology template; and
a validation unit configured to declare the potential ST segment shift to be an actual ST segment shift based on the morphology indicator, the ST episode detection unit configured to declare an ST episode when a predetermined number of actual ST segment shifts are validated.

11. The system of claim 10, wherein the template acquisition unit is configured to obtain first and second reference morphology templates, the first reference morphology template based on at least one baseline cardiac signal associated with a normal physiology waveform, the second reference morphology template based on at least one baseline cardiac signal associated with an abnormal physiology waveform.

12. The system of claim 10, wherein the comparison unit is configured to compare the cardiac signals to the first and second reference morphology templates to derive first and second morphology indicators, respectively.

13. The systems of claim 10, wherein the validation unit is configured to declare the potential ST segment shift to be an actual ST segment shift based on the first and second morphology indicators.

14. The system of claim 10, wherein the template acquisition unit is configured to collect a reference morphology template based on sampling of multiple baseline cardiac signals associated with normal physiology waveforms.

15. The system of claim 10, wherein the sensing, identifying, comparing and declaring functions are repeated for sets of cardiac events, each cardiac event associated with a heart beat, the ST episode detection unit configured to declare a set of the cardiac events to exhibit ST segment shift when a predetermined number of cardiac events in the corresponding set have morphology indicators indicating a predetermined degree of match with the reference morphology template.

16. The system of claim 10, wherein the template acquisition unit is configured to collect multiple reference morphology templates, each of which is associated with a unique corresponding heart rate zone, the identifying, comparing and declaring operations utilizing one of the multiple reference templates corresponding to a present heart rate associated with the cardiac signals sensed.

17. The system of claim 10, wherein the comparison unit is configured to identify a polarity of a dominant R-peak in the cardiac signal, and compare the polarity of the dominant R-peak in the cardiac signal with a polarity of a dominant R-peak in the reference morphology template, the validation unit configured to validate the actual ST segment shift based in part on the polarity comparison.

18. The system of claim 10, wherein the comparison unit is configured to compare one or more of the following: wave frequency, an area of each QRS segment, a sequence of R-peaks, a number of R-peaks, an amplitude of R-peaks, and a polarity of the R-peaks.
